(19) United States
(12) Patent Application Publication
(10) Pub. No.: US 2014/0046086 A1
(43) Pub. Date: Feb. 13, 2014

(54) PROCESS FOR THE PREPARATION OF TAFLUPROST AND INTERMEDIATES THEREOF

(71) Applicant: Scinopharm (Changshu) Pharmaceuticals, Ltd., Changshu (CN)

(72) Inventor: Wen-Hsien Wen, Changshu (CN)

(73) Assignee: Scinopharm (Changshu) Pharmaceuticals, Ltd., Changshu (CN)

(21) Appl. No.: 13/962,058

(22) Filed: Aug. 8, 2013

Related U.S. Application Data
(60) Provisional application No. 61/681,905, filed on Aug. 10, 2012.

(51) Int. Cl. C07C 69/007; C07C 67/307; C07F 5/04

(52) U.S. Cl. CPC C07C 69/007; C07F 5/04; C07C 67/307
USPC 558/288; 560/53; 560/63

(57) ABSTRACT

The present invention provides efficient, economical and environmental friendly methods for synthesis of prostaglandin analogs including tafluprost and intermediates thereof. The invention involves a selective oxidation using in situ boronate ester protection and a unique crystallization method to remove the undesired isomers of fluorinated intermediates.

### CROSS-REFERENCES TO RELATED APPLICATIONS

This application claims priority from U.S. Provisional Patent Application Ser. No. 61/681,905, filed on Aug. 10, 2012, the entire content of which is hereby incorporated by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Tafluprost (Taflotan™ or Saflutan™) is a prostaglandin analog used to control the progression of glaucoma and to manage ocular hypertension. It reduces intraocular pressure by increasing the outflow of aqueous fluid from the eyes. Tafluprost was developed by Asahi Glass Company Ltd. and Santen Pharmaceutical Co., Ltd. The commercial product, ZIOPTAN™, is a preservative-free, single-dose formulation containing 15 tafluprost at 0.3 mL per dose and was approved by the FDA in early 2012.

### Synthesis of fluorine-containing prostaglandin derivatives and intermediates thereof was disclosed in U.S. Pat. No. 5,886,035. A more detailed description of a process for preparation of tafluprost from a Corey lactone starting material was subsequently disclosed (Tetrahedron letters, 2004, 45, 1527-1529).

FIG. 1 illustrates the known synthetic route to tafluprost from Corey lactone 1. The lactone is converted to tafluprost over six steps including: i) a Horner-Emmons reaction with phosphonate 2; ii) difluorination with morpholinosulfur trifluoride; iii) removal of the benzoyl group with potassium carbonate; iv) reduction with diisobutylaluminum hydride; v) a Wittig reaction with an ylide prepared from 4-carboxybutyltriphenylphosphonium bromide; and vi) esterification with isopropyl iodide and 1,8-diazabicyclo[5.4.0]undec-7ene (DBU). Disadvantages of this process include the high cost of the lactone starting material, the column chromatography and other costly work-up procedures required at every step in the process, and the carcinogenicity of the isopropyl iodide used in the final step of the process (reported by Poirier, et al., 1975).

Other known routes to prostaglandin analogs include a process reported by Hacksell, et al., as shown in FIG. 2 (J. Org. Chem. 1996, 61, 4028-4034). The C9-OH and the C11-OH of the triol starting material were protected in situ using phenylboronic acid prior to oxidation with pyridinium chlorochromate (PCC)/Al₂O₃ to provide an aldehyde intermediate. Notably, the oxidized product is an aldehyde in the Hacksell procedure as compared to the ketone resulting from the methods of the present invention, set forth below.

Another route, disclosed in WO 2009/136281A1 and shown in FIG. 3, includes in situ protection of the C9-OH and the C11-OH of bimatoprost with butylboronic acid prior to esterification of the C15-OH. The same publication discloses oxidation of the bimatoprost C15-OH using 2,3-dichloro-5,6-dicyano-1,4-benzoquinone (DDQ) without protection of the C9-OH and the C11-OH, as shown in FIG. 4. However, DDQ is used in large excess and is difficult to remove. DDQ is also highly toxic, which limits the practicality of the method.

As such, there is a strong demand for a convenient process that is suitable for preparation of tafluprost and related intermediates on a commercial scale with high purity and without complicated and costly purification steps. The present invention addresses this and other needs.

### BRIEF SUMMARY OF THE INVENTION

In a first aspect, the invention provides a process for preparing a prostaglandin analog of formula I

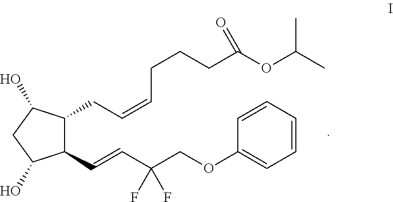 [SMILES: CC(C)OC(=O)CCC/C=C\C[C@@H]1[C@@H](/C=C/C(F)(F)COc2ccccc2)[C@H](O)C[C@@H]1O]

The process includes:
a) contacting a compound of formula II

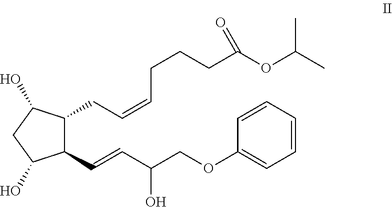 [SMILES: CC(C)OC(=O)CCC/C=C\C[C@@H]1[C@@H](/C=C/C(O)COc2ccccc2)[C@H](O)C[C@@H]1O]

with a compound selected from the group consisting of a boronic acid, a boronate ester, and an aminoborane, under conditions sufficient to provide a compound of formula III

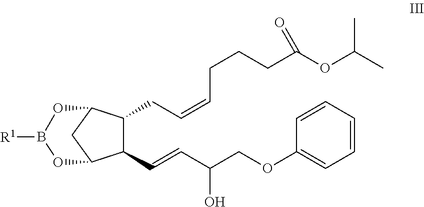

wherein R¹ is an optionally substituted alkyl group, an optionally substituted aryl group, or a polystyrene support;
b) converting the compound of formula III to a compound of formula V

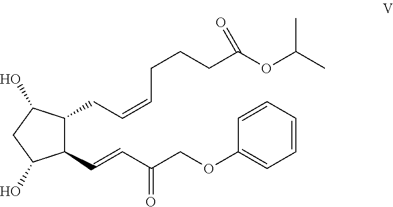 [SMILES: CC(C)OC(=O)CCC/C=C\C[C@@H]1[C@@H](/C=C/C(=O)COc2ccccc2)[C@H](O)C[C@@H]1O]

using an oxidant and a subsequent basic wash solution;
c) converting the compound of formula V to a compound of formula VI

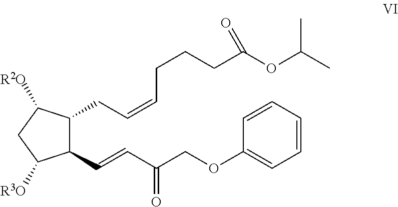

wherein R² and R³ are independently selected from hydroxy protecting groups or are taken together to form a single hydroxy protecting group;
d) fluorinating the compound of formula VI to give a compound of VII

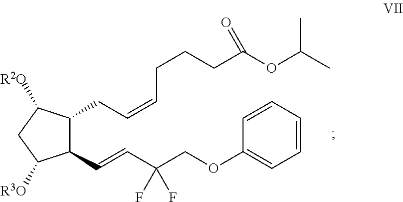

and
e) removing R² and R³ from the compound of VII under basic conditions to provide the compound of formula I.

In a second aspect, the invention provides novel compounds including those according to structures IIIa, VI and VII:

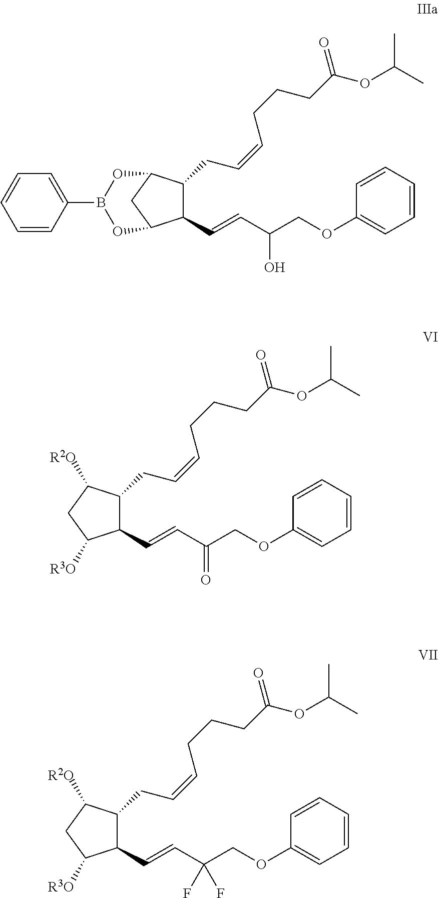

wherein R² and R³ are independently selected from hydroxy protecting groups or are taken together to form a single hydroxy protecting group.

### BRIEF DESCRIPTION OF THE DRAWINGS

FIG. 1 shows a route to fluorine-containing prostaglandin derivatives disclosed in U.S. Pat. No. 5,886,035.

FIG. 2 shows a route to prostaglandin disclosed by Hacksell, et al. J. Org. Chem. 1996, 61, 4028-4034, using phenylboronic acid followed by pyridinium chlorochromate oxidation.

FIG. 3 shows a route to bimatoprost derivatives disclosed in WO 2009/136281.

FIG. 4 shows the selective oxidation of the bimatoprost C15 hydroxyl group using DDQ as disclosed in WO 2009/136281.

FIG. 5 shows the synthetic route to prostaglandin analogs as developed by the present applicant in U.S. Pat. No. 7,897,795.

FIG. 6 shows the synthetic route to a compound of formula II as disclosed by the present applicant.

FIG. 7 shows the process for preparation of tafluprost according to the methods of the present invention.

FIG. 8 shows the selective oxidation of the prostaglandin C15-hydroxyl group using in situ boronate ester protection of cis diol intermediates.

FIG. 9 shows the oxidation of cis diol II and trans diol II′ to acquire compound V with high optical purity.

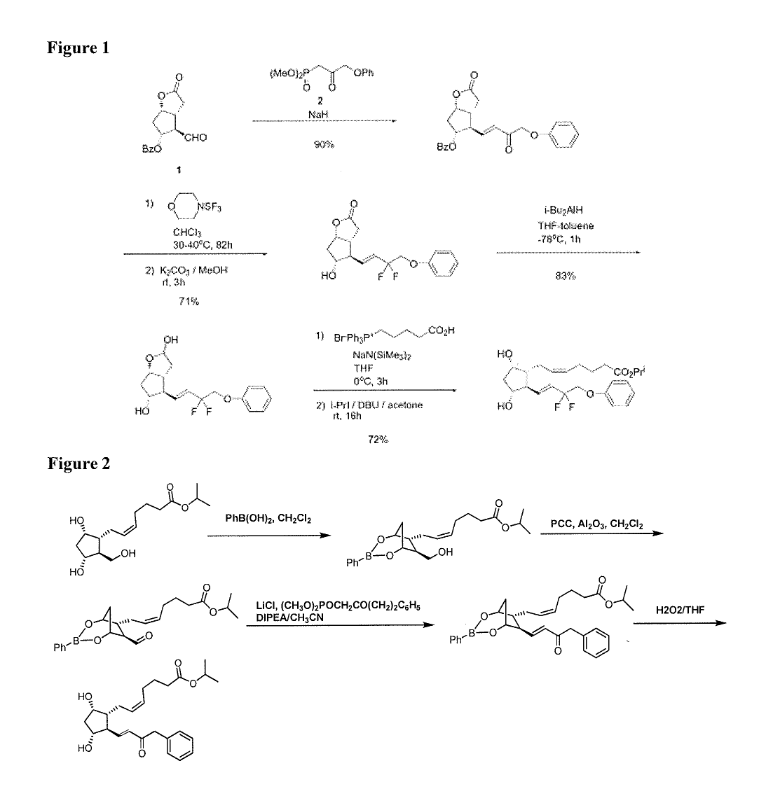

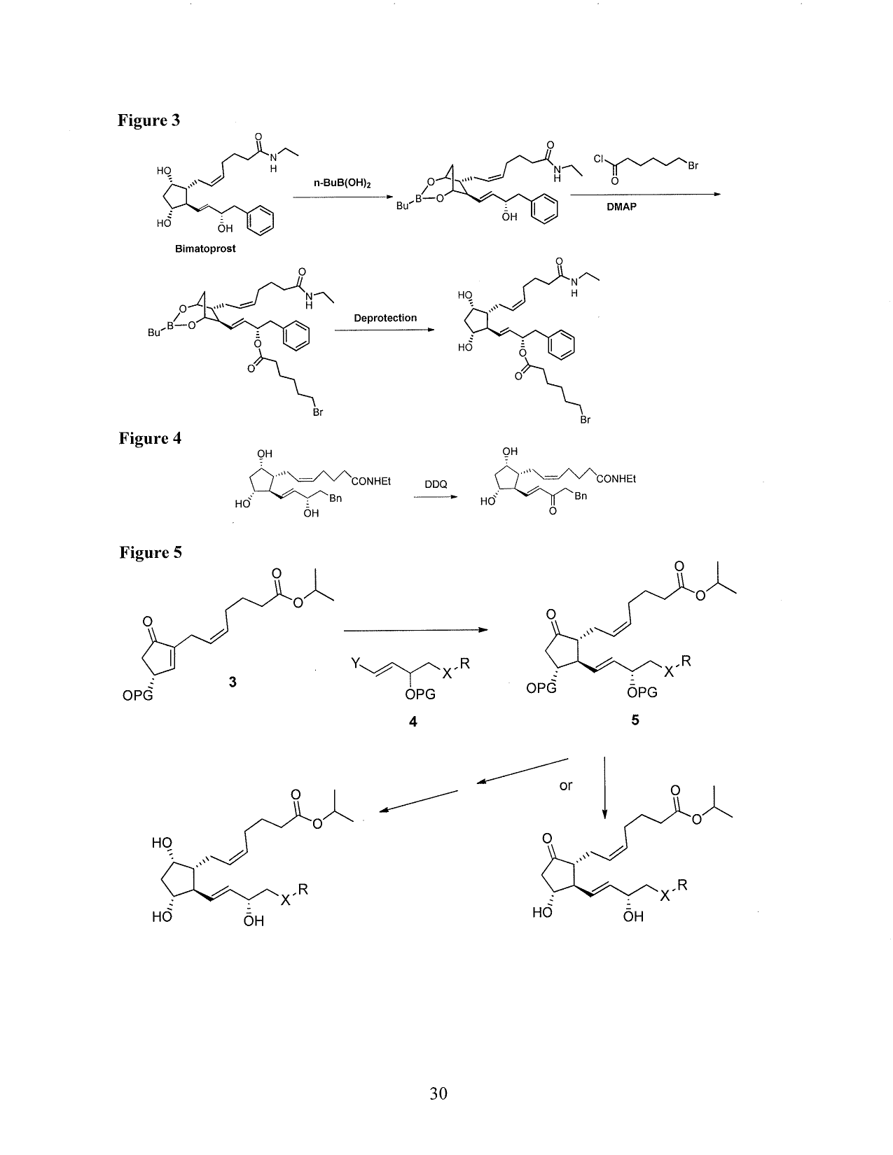

### DETAILED DESCRIPTION OF THE INVENTION

### I. General

The present invention provides a process for preparation of prostaglandin analogs. A novel, selective oxidation via in situ boronate ester protection of the cis diol starting materials has been discovered to be mild, safe, economically efficient, and environmentally friendly. The inventive process avoids the use of toxic and unstable DDQ. The products of the oxidation route can be easily purified without lengthy column chromatography. Advantageously, undesired trans diol isomers can be easily removed from the desired product using the inventive methods. In some embodiments, large aromatic hydroxyl protecting groups allow for crystallization and isolation of key intermediates, in contrast with other prostaglandin analogs and intermediates that are frequently liquids or oils. Crystallization is less expensive, more efficient and environmentally friendly than column chromatography for preparation of these compounds on an industrial scale.

### II. Definitions

As used herein, the term “contacting” refers to the process of bringing into contact at least two distinct species such that they can react. It should be appreciated, however, that the resulting reaction product can be produced directly from a reaction between the added reagents or from an intermediate from one or more of the added reagents that can be produced in the reaction mixture.

As used herein, the term “alkyl” by itself or as part of another substituent, means, unless otherwise stated, a straight or branched chain hydrocarbon radical. Alkyl substituents, as well as other hydrocarbon substituents, may contain number designators indicating the number of carbon atoms in the substituent (i.e. C₁-C₈ means one to eight carbons), although such designators may be omitted. Unless otherwise specified, the alkyl groups of the present invention contain 1 to 12 carbon atoms. For example, an alkyl group can contain 1-2, 1-3, 1-4, 1-5, 1-6, 1-7, 1-8, 1-9, 1-10, 1-11, 1-12, 2-3, 2-4, 2-5, 2-6, 3-4, 3-5, 3-6, 4-5, 4-6 or 5-6 carbon atoms. Examples of alkyl groups include methyl, ethyl, n-propyl, isopropyl, n-butyl, t-butyl, isobutyl, sec-butyl, n-pentyl, n-hexyl, n-heptyl, n-octyl, and the like.

As used herein, the term “boronic acid” refers to an alkyl or aryl substituted boric acid containing a carbon-boron bond. Examples of boronic acids include, but are not limited to, alkyl boronic acids, phenyl boronic acids, polymer-supported boronic acids, and diboronic acids.

As used herein, the term “boronate ester” refers to an ester that can result from the reaction of a boronic acid with an alcohol. One of skill in the art will appreciate that boronate esters are not necessarily formed by such a reaction, but can be prepared by a variety of methods.

As used herein, the term “aminoborane” refers to a borane derivative containing at least two boron-nitrogen bonds.

As used herein, the term “protecting group” refers to a moiety that is formed to render a functional moiety unreactive. The protecting group can be removed so as to restore the functional moiety to its original state. Various protecting groups and protecting reagents, including hydroxy protecting groups, are well known to one of ordinary skill in the art and include compounds that are disclosed in Protective Groups in Organic Synthesis, 4th edition, T. W. Greene and P. G. M. Wuts, John Wiley & Sons, New York, 2006, which is incorporated herein by reference in its entirety.

### III. Embodiments of the Invention

The present invention provides a process for preparing a prostaglandin analog of formula I

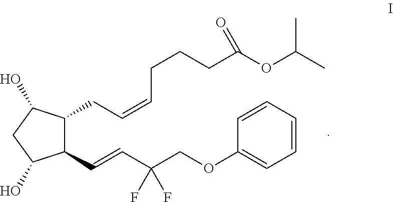 [SMILES: CC(C)OC(=O)CCC/C=C\C[C@@H]1[C@@H](/C=C/C(F)(F)COc2ccccc2)[C@H](O)C[C@@H]1O]

The process includes:

a) contacting a compound of formula II

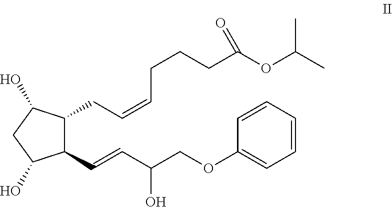 [SMILES: CC(C)OC(=O)CCC/C=C\C[C@@H]1[C@@H](/C=C/C(O)COc2ccccc2)[C@H](O)C[C@@H]1O]

with a compound selected from the group consisting of a boronic acid, a boronate ester, and an aminoborane, under conditions sufficient to provide a compound of formula III,

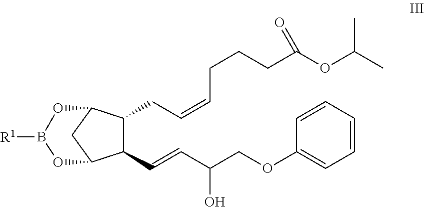

wherein R¹ is an optionally substituted alkyl group, an optionally substituted aryl group, or a polystyrene support;

b) converting the compound of formula III to a compound of formula V

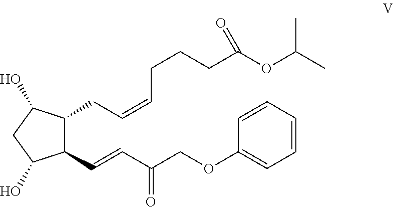 [SMILES: CC(C)OC(=O)CCC/C=C\C[C@@H]1[C@@H](/C=C/C(=O)COc2ccccc2)[C@H](O)C[C@@H]1O]

using an oxidant and a subsequent basic wash solution;

c) converting the compound of formula V to a compound of formula VI

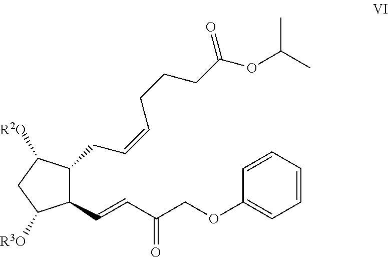

wherein R² and R³ are independently selected from hydroxy protecting groups or are taken together to form a single hydroxy protecting group;

d) fluorinating the compound of formula VI to give a compound of VII

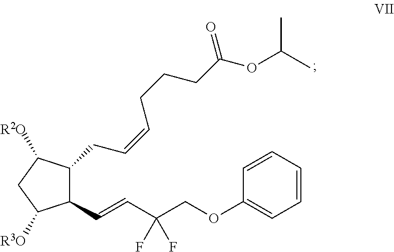

and

e) removing R² and R³ from the compound of VII under basic conditions to provide the compound of formula I.

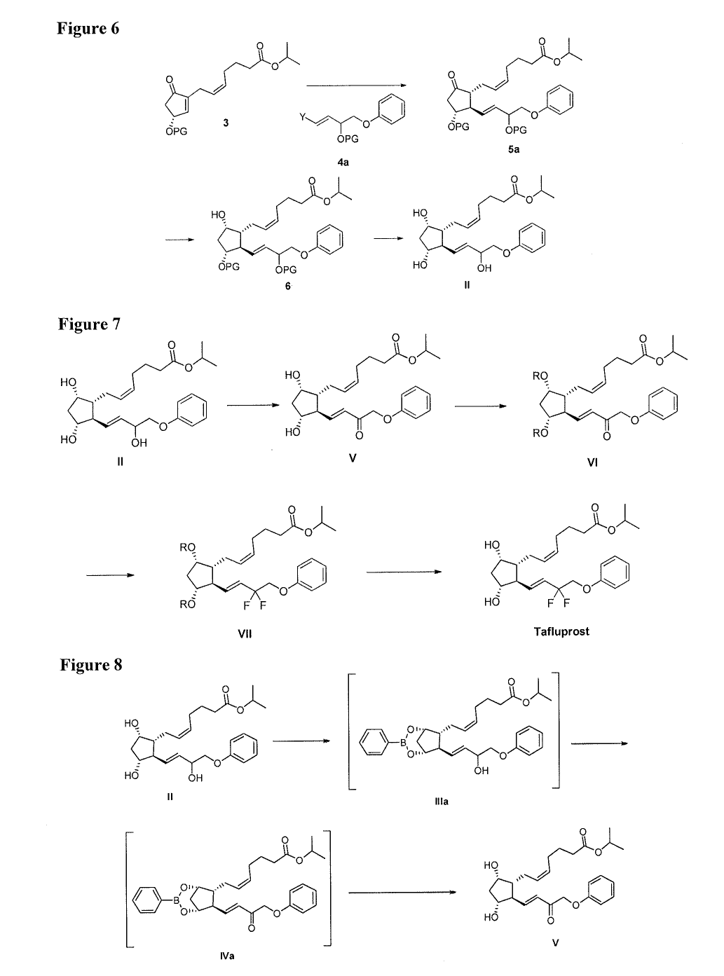

The inventive process utilizes compound II, which is readily prepared via a method for synthesis of prostaglandin analogs previously disclosed by the applicant (see, U.S. Pat. No. 7,897,795). The synthetic route is shown in FIG. 5. In this method, an intermediate 3 reacts with cuprate compound 4 to give a compound 5. The compound 5 can be optionally modified and deprotected to provide various prostaglandin analogs. The present inventors envisioned a method for synthesis of tafluprost and other prostaglandin analogs utilizing compound II as a key intermediate. The inventive route is shown in FIG. 6. Compound II is prepared via conjugate addition of compound 3 with cuprate 4a, followed by selective reduction and deprotection. Unlike previous methods employing optically pure 4 to construct compound 5, the present cuprate 4a is racemic. After compound 5a is constructed, it is reduced and deprotected to acquire compound II.

In some embodiments, the process includes synthesis of tafluprost according to FIG. 7. In the inventive process, either the (R)— or (S)—C15-OH can be oxidized to provide a ketone V. Diol protection provides a compound VI (where each R group is an independently selected protecting group) that can be converted to a compound VII through difluorination. Finally, deprotection of VII affords tafluprost.

In some embodiments, the inventive process includes a selective oxidation reaction as exemplified in FIG. 8. The process can include reaction of a boronic acid with compound II to form an intermediate III. In some embodiments, R¹ of intermediate III is selected from unsubstituted alkyl, phenyl and a polystyrene support. A mono-substituted boronic acid (B(OH)₂R¹) provides bi-dentate protection to the C9 and C11 cis hydroxy groups, forming the six-membered ring present in intermediate III, as well as in compounds IIIa and IVa shown in FIG. 8. Any suitable mono-substituted boronic acid may be used in the present invention. In some embodiments, the boronic acid is selected from an alkyl boronic acid, a phenyl boronic acid, a polymer-supported boronic acid, and a diboronic acid. For boronic acids having a formula (B(OH)₂R¹), R¹ can be, but not limited to, substituted or unsubstituted phenyl, methyl, ethyl, propyl, butyl, pentyl, hexyl, cyclohexyl, thionyl, and a polystyrene support. In the case of compound IIIa, R¹ is phenyl. One of skill in the art will appreciate that still other mono-substituted boronic acids may be useful in the inventive process.

In some embodiments, the compound of formula III can be formed from compound II via a transesterification with a boronate ester. In some embodiments, the boronate ester is selected from an alkyl dialkoxyl borane, a dialkyl phenyl boronate, a trialkyl boronate, a 1,1,2,2-tetraalkoxy-diborane, and a polystyrene-supported dialkyl boronate. The alkyl groups can be independently selected from methyl, ethyl, propyl, butyl, pentyl, hexyl, and cyclohexyl.

In some embodiments, the compound of formula III can be formed from compound II via reaction with an aminoborane. The aminoborane can be, for example, a tris(dialkylamino)borane, an alkyldiaminoborane or a tetrakis(dialkylamino)diboron.

The oxidative conversion of compound IIIa to compound IVa can be conducted under a variety of conditions. In some embodiments, conversion of compound IIIa includes using an oxidant and a basic wash solution. The oxidant can be, but is not limited to, pyridinium chlorochromate (PCC); PCC/Al₂O₃; chromium oxidants including Jones reagent, Collins reagent, and pyridinium dichromate (PDC); dimethyl sulfoxide (DMSO)-based systems including DMSO/dicyclohexylcarbodiimide (DCC), DMSO/sulfur trioxide pyridine complex (SO₃-pyr), DMSO/oxalyl chloride (COCl)₂, DMSO/trifluoroacetic anhydride (TFAA), and DMSO/acetic anhydride (Ac₂O); Dess-Martin periodinane; 2-iodoxybenzoic acid (IBX); and (2,2,6,6-tetramethyl-piperidin-1-yl)oxyl (TEMPO). In some embodiments, the oxidant is selected from PCC, PCC/Al₂O₃, Jones reagent, Collins reagent, PDC, DMSO/DCC, DMSO/SO₃-pyridine, DMSO/(COCl)₂, DMSO/TFAA, DMSO/Ac₂O, Dess-Martin periodinane, IBX, and TEMPO. In some embodiments, the oxidant is PCC/Al₂O₃. PCC/Al₂Cl₃ can be simply removed via filtration.

The removal of boronate ester protecting groups can be conducted under any suitable conditions. In some embodiments, protecting groups are removed by oxidative cleavage using hydrogen peroxide (H₂O₂). In some embodiments, protecting groups are removed via hydrolysis using acid or base. In some embodiments, protecting groups are removed using a basic wash solution. In some embodiments, the basic wash solution is selected from NaOH (aq), Na₂CO₃ (aq), NaHCO₃ (aq), K₂CO₃ (aq), LiOH (aq), and KOH (aq). In particular, aqueous sodium hydroxide can readily remove boronate ester protecting groups without affecting other functional groups.

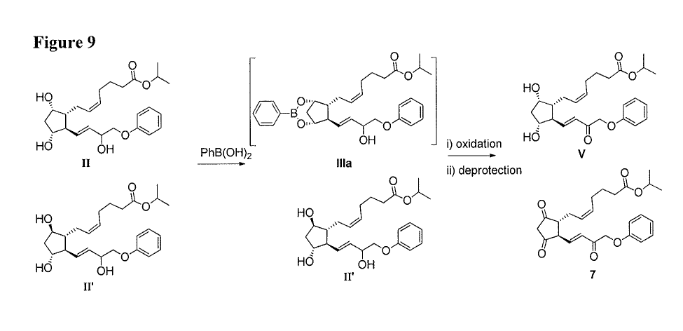

In a related aspect, the present invention provides a method to resolve the cis diol II from its trans counterpart II′, as shown in FIG. 9. Use of a bulky hydride such as L-selectride, N-selectride and K-selectride could be used to give the desired II as the major product. However, this leads to formation of II′ which is difficult to remove, even via column chromatography. In the present invention, the cis C9- and C11-hydroxy groups of diol II can be protected with a mono-substituted boronic acid while trans C9- and C11-hydroxy groups of diol II′ remain unprotected. The cis diol II can react with phenylboronic acid to construct a 6-member ring readily, but the trans isomer II′ is unable to do this due to ring strain. This leads to oxidation of the unprotected trans diol in the subsequent step. Therefore the undesired compound II′ can be converted to the over-oxidized product 7 which can easily be separated from compound V. Using the inventive process, in situ boronate ester protection followed by oxidation and deprotection circumvents the difficult separation of the trans diol II′ from compound V, affording the desired product in highly pure form without further purification. Notably, epimer separation is not utilized in this fashion when using the procedure of Hacksell as described above.

The inventive process includes protecting compound V to form compounds of formula VI. The resulting protecting groups can include R² moieties and R³ moieties at the C9 and C11 hydroxyl groups. In some embodiments, R² and R³ are independently selected from acetate, chloroacetate, dichloroacetate, trichloroacetate, trifluoroacetate, pivaloate, benzoate, p-methoxybenzoate, p-bromobenzoate, p-chlorobenzoate, Fmoc, Cbz, 2-naphthalenecarboxylate and 4-phenyl benzoate.

The inventive process includes preparing a compound VII from a compound VI by difluorination. Any suitable fluorinating agent can be used in the process. For example, the fluorinating agent can be a commercially available fluorinating agent such as diethylaminosulfur trifluoride (DAST), bis(2-methoxyethyl)aminosulfur trifluoride (Deoxofluor), diethylaminodifluorosulfinium tetrafluoroborate (Xtalfluor-E™), morpholinoifluorosulfinium tetrafluoroborate (Xtalfluor-M™), 4-tert-Butyl-2,6-dimethylphenylsulfur trifluoride (Fluolead™), and the like. In some instances, the application of such agents in the oxidation of a compound VII can lead to the formation of three to four isomeric impurities with the same molecular weights that cannot be readily removed via column chromatography. In these instances, it is believed that these isomeric impurities resulted from the 1,3-difluorination of compounds VI. In order to circumvent this purity issue, a purification method based on crystallization of compounds VI and compounds VII has been developed.

As shown in Table 1, six different protecting groups were employed for compounds VII; in particular, compounds VIIa to VIIf were synthesized. Notably, tafluprost and its intermediates are typically oils. In fact, the benzoyl, 4-bromobenzoyl, 4-methoxybenzoyl and acetyl derivatives prepared via the methods of the present invention were oils. Surprisingly, 4-phenylbenzoyl derivative VIIa and 2-naphthalcarbonyl (2-NaphCO) derivatives VIIb were isolated as crystalline solids (see Table 1). These solid intermediates of tafluprost can be conveniently purified through crystallization without the burden of chromatography.

TABLE 1
Physical Characteristics of Prostaglandin Intermediates

| Protecting group (R², R³) | VII | Physical State |
|---|---|---|
| PhBz | VIIa | Solid |
| 2-NaphCO | VIIb | Solid |
| Bz | VIIc | Oil |
| 4-BrBz | VIId | Oil |
| 4-MeOBz | VIIe | Oil |
| Ac | VIIf | Oil |

VIIa showed higher melting point (70-71° C.) than VIIb (53-54° C.), which indicated VIIa is better for performing crystallization study. Therefore, 4-phenylbenzoyl derivatives VIIa are preferably used in the preparation of Tafluprost.

Table 2 indicated that column chromatography could not remove the isomeric impurities effectively, especially impurity A. The best fraction from column is fraction 7 where there was only 95.7% purity of VIIa and 2.08% impurity A was still left. HPLC result also showed that the peak of impurity A located very closely with VIIa. By contrast, the purity of VIIa could be improved to 99.0% through crystallization and impurities reduced to 0.13%, 0.57%, and 0.25%.

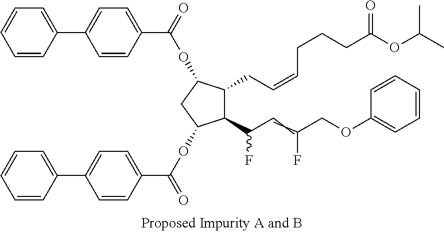 [SMILES: CC(C)OC(=O)CCC/C=C\C[C@@H]1[C@@H](C(F)C=C(F)COc2ccccc2)[C@H](OC(=O)c2ccc(-c3ccccc3)cc2)C[C@@H]1OC(=O)c1ccc(-c2ccccc2)cc1]

TABLE 2
Impurity Profile of VIIa After Crystallization and column chromatography

| | | | | | Impurity C |
|---|---|---|---|---|---|
| Purification method | Solvent | VIIa | Impurity A | Impurity B | (unknown) |
| Column chromatography | | | | | |
| Fraction 1 | EtOAc/n-Heptane | 84.8% | 7.22% | 0.62% | 2.84% |
| Fraction 7 | EtOAc/n-Heptane | 95.7% | 2.08% | 0.35% | 0.55% |
| Fraction 19 | EtOAc/n-Heptane | 71.8% | 22.3% | 2.93% | 0.0% |
| Crystallization | | | | | |
| 1^{st} crystallization | THF/n-Heptane | 96.4% | 1.43% | 1.32% | 0.78% |
| 2^{nd} crystallization | THF/n-Heptane | 97.7% | 0.84% | 0.93% | 0.38% |
| 3^{rd} crystallization | THF/n-Heptane | 98.2% | 0.53% | 0.65% | 0.39% |
| 4^{th} crystallization | Toluene/n-Heptane | 99.0% | 0.13% | 0.57% | 0.25% |

In a related aspect, the present invention provides compounds that are useful for the synthesis of tafluprost and other prostaglandin analogs. In some embodiments, the present invention provides a compound of formula IIIa

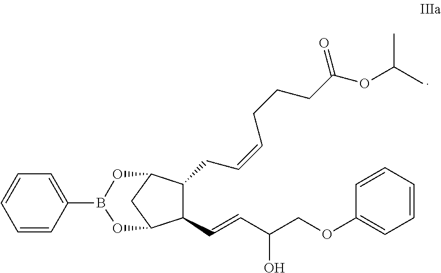 [SMILES: CC(C)OC(=O)CCC/C=C\C[C@@H]1[C@@H](/C=C/C(O)COc2ccccc2)[C@H]2C[C@@H]1OB(c1ccccc1)O2]

In some embodiments, the present invention provides a compound of formula VI

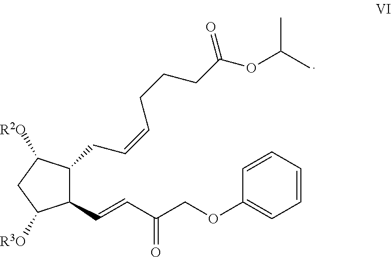

wherein R² and R³ are independently selected from hydroxy protecting groups or are taken together to form a single hydroxy protecting group.

In some embodiments, R² and R³ are independently selected from the group consisting of acetate, chloroacetate, dichloroacetate, trichloroacetate, trifluoroacetate, pivaloate, benzoate, p-methoxybenzoate, p-bromobenzoate, p-chlorobenzoate, Fmoc, Cbz, 2-naphthalenecarboxylate and 4-phenyl benzoate. In some embodiments, R² and R³ are 4-phenyl benzoate.

In some embodiments, the present invention provides a compound of formula VII

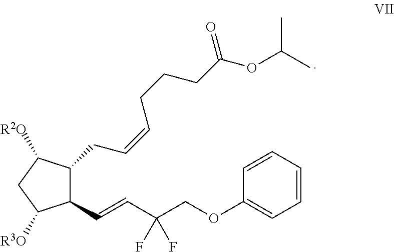

wherein R² and R³ are independently selected from hydroxy protecting groups or are taken together to form a single hydroxy protecting group.

In some embodiments, R² and R³ are independently selected from the group consisting of acetate, chloroacetate, dichloroacetate, trichloroacetate, trifluoroacetate, pivaloate, benzoate, p-methoxybenzoate, p-bromobenzoate, p-chlorobenzoate, Fmoc, Cbz, 2-naphthalenecarboxylate and 4-phenyl benzoate. In some embodiments, R² and R³ are 4-phenyl benzoate.

In some embodiments, the compound of formula VIIa contains impurities of less than 2%. In some embodiments, the impurities contained with the compound of formula VIIa are below 1%.

In some embodiments, tafluprost is prepared from compound VIIa via hydrolysis. Hydrolysis can be conducted under basic conditions. The base can be, for example, potassium carbonate, sodium hydroxide, guanidine, sodium isopropoxide, and the like. One of skill in the art will understand that a variety of bases can be applied in the process. In some embodiments, hydrolysis is conducted using a solution of sodium isopropoxide in an alcohol. In some embodiments, hydrolysis is conducted using a solution of sodium isopropoxide in isopropyl alcohol.

### IV. Examples

The following examples are presented to illustrate, but not limit, certain aspects of the present invention.

Abbreviations used in the Examples include:

DDQ, 2,3-dichloro-5,6-dicyano-1,4-benzoquinone; DCM, dichloromethane; PCC, pyridinium chlorochromate; MTBE, methyl t-butyl ether; h, hour; MHz, megahertz; DMAP, 4-dimethylaminopyridine; THF, tetrahydrofuran; MS, mass spectrometry; NMR, nuclear magnetic resonance; IPA, isopropyl alcohol; AcOH, acetic acid; EtOAc, ethyl acetate.

### Example 1

### Preparation of (Z)-isopropyl 7-((1R,2R,3R,5S)-3,5-dihydroxy-2-((E)-3-oxo-4-phenoxybut-1-en-1-yl)cyclopentyl)hept-5-enoate (V) without Boronate Ester Protection

To a solution of compound II (7.35 g, 1 eq.) in CH₂Cl₂/1,4-dioxane (v/v=1:1, 40 ml) was added DDQ (11.57 g, 3 eq.) at 25° C. The mixture was warmed to 40° C. and stirred for 11 h, then cooled to 25° C. prior to addition of saturated NaHCO₃ (20 mL). After filtration of the mixture, the filtrate was extracted with EtOAc (200 mL). The separated organic layer was washed with saturated twice with NaHCO₃ (100 mL) and then with brine. The solvent was evaporated to provide the crude compound. Purification through SiO₂ with EtOAc/Heptane (v/v=2:1) afforded purified compound V (4.8 g, 65%) as pale-yellow oil. ¹H NMR (400 MHz, Chloroform-d): δ 7.37-7.26 (m, 2H), 7.06-6.87 (m, 4H), 6.54 (d, J=15.6 Hz, 1H), 5.45-5.30 (m, 2H), 5.02 (hept, J=6.2 Hz, 1H), 4.73 (d, J=0.8 Hz, 2H), 4.26 (t, J=4.3 Hz, 1H), 4.19-4.07 (m, 1H), 2.60 (td, J=9.8, 4.2 Hz, 1H), 2.42-2.24 (m, 3H), 2.21-2.01 (m, 6H), 1.96-1.87 (m, 1H), 1.77-1.61 (m, 3H), 1.26 (s, 3H), 1.24 (s, 3H).

### Example 2

### Preparation of (Z)-isopropyl 7-((1R,2R,3R,5S)-3,5-dihydroxy-2-((E)-3-oxo-4-phenoxybut-1-en-1-yl)cyclopentyl)hept-5-enoate (V) with In Situ Formation of Boronate ester protecting groups and pyridinium chlorochromate as an oxidant.

### Preparation of PCC/Al₂O₃

To a solution of PCC (450 mg, 3 eq) in acetone (5 mL) was added Al₂O₃ (1.35 g). The reaction mixture was stirred at 25±5° C. for 5 min and concentration to provide the PCC-Al₂O₃ complex (1.8 g).

### (Z)-isopropyl 7-((1R,5S,6R,7R)-7-((E)-3-hydroxy-4-phenoxybut-1-en-1-yl)-3-phenyl-2,4-dioxa-3-borabicyclo[3.2.1]octan-6-yl)hept-5-enoate (IIIa)

To a flask charged with phenylboronic acid (170 mg, 1.4 mmol) was added a solution of II (300 mg, 0.7 mmol) in toluene (50 mL). The solution was warmed to reflux to remove water with Dean-Stark apparatus. After 2 h, the mixture was cooled down and concentrated to provide crude compound IIIa.

### Preparation of Compound V Via Oxidation and Deprotection of Compound IIIa

The above-prepared compound IIIa was dissolved in CH₂Cl₂ (4.5 mL) and then added PCC-Al₂O₃ (w/w=1/3, 1.8 g) at 25±5° C., the mixture was stirred at 25±5° C. for 3 h, the resulting mixture was filtered through a short plug of Celite and washed with MTBE (10 mL). The filtrate was washed with 1% NaOH (10 mL) twice to provide crude compound V (220 mg, 73%) with 75% purity.

### Example 3

### Preparation of (Z)-isopropyl 7-((1R,2R,3R,5S)-3,5-dihydroxy-2-((E)-3-oxo-4-phenoxybut-1-en-1-yl)cyclopentyl)hept-5-enoate (V) with In Situ Formation of Boronate Ester Protecting Groups and Pyridine Sulfur Trioxide as an Oxidant

To a solution of compound IIIa (4.0 g, 9.2 mmol) in toluene (20 mL) was added DIPEA (7.13 g) and a solution of pyridine sulfur trioxide (3.52 g, 22 mmol) in DMSO (12.25 mL) at 0° C. After 4 h, the reaction was quenched with H₂O (40 mL) and extracted with MTBE (60 mL). The organic layer was washed with 1% NaOH (10 mL) twice to provide crude compound V (2.4 g, 60%).

### Example 4

### Preparation of (Z)-isopropyl 7-((1R,2R,3R,5S)-3,5-dihydroxy-2-((E)-3-oxo-4-phenoxybut-1-en-1-yl)cyclopentyl)hept-5-enoate (V) with In Situ Formation of Boronate Ester Protecting Groups and Dess-Martin Periodinane as an Oxidant

To a solution of IIIa (0.5 g) in CH₂Cl₂ was added Dess-Martin periodinane (0.81 g) followed by stirring at 25° C. for 4 h. The reaction was quenched with sat. NaHCO₃ (5 mL) and separated by adding H₂O (5 mL), and CH₂Cl₂ (5 mL). The aqueous layer was extracted with CH₂Cl₂ (10 mL). The combined organic layer was washed with sat. NH₄Cl (10 mL) and brine (10 mL) followed by evaporation to furnish crude compound V (0.40 g, 97%).

### Example 5

### Preparation of (1S,2R,3R,4R)-3-[(1E)-3-oxo-4-phenoxybut-1-en-1-yl]-2-[(2Z)-7-oxo-7-(propan-2-yloxy)hept-2-en-1-yl]-4-[(4-phenylphenyl)carbonyloxy]cyclopentyl 4-phenylbenzoate (VIa)

A solution of compound V (1.0 g, 2.3 mmol) in CH₂Cl₂ (3 mL) was added drop-wise to a solution of PhBzCl (2.0 g, 9.3 mmol) in pyridine (3 mL) at 5±5° C. The mixture was stirred at 25±5° C. until compound V was consumed completely (2 h). The mixture was separated by adding MTBE (10 mL) and H₂O (10 mL). The organic layer was washed twice with 2 N HCl (10 mL), followed by H₂O (10 mL) and brine (10 mL). The organic layer was dried over MgSO₄, filtered, evaporated, and purified by chromatography (eluent: EtOAc/Heptane=1/4) to afford compound VIa (1.6 g, 91%). ¹H NMR (400 MHz, Chloroform-d): δ 8.17 (d, J=8.4 Hz, 2H), 7.97 (d, J=8.4 Hz, 2H), 7.70 (d, J=8.4 Hz, 2H), 7.64 (dd, J=8.2, 1.5 Hz, 2H), 7.58-7.35 (m, 10H), 7.30 (dd, J=8.7, 7.3 Hz, 2H), 7.16 (dd, J=15.8, 8.8 Hz, 1H), 6.99 (tt, J=7.3, 1.1 Hz, 1H), 6.93 (dt, J=7.7, 1.1 Hz, 2H), 6.67 (dd, J=15.7, 0.9 Hz, 1H), 5.53 (t, J=4.6 Hz, 1H), 5.44-5.35 (m, 3H), 4.96 (hept, J=6.2 Hz, 1H), 4.78 (d, J=1.4 Hz, 2H), 3.17-3.05 (m, 1H), 2.71 (ddd, J=15.9, 8.3, 5.0 Hz, 1H), 2.49-2.36 (m, 1H), 2.30-2.16 (m, 2H), 2.16-2.05 (m, 3H), 2.03-1.88 (m, 2H), 1.68-1.49 (m, 2H), 1.19 (d, J=1.2 Hz, 3H), 1.18 (d, J=1.3 Hz, 3H). MS: m/z 808.3 [M+NH₄]⁺.

### Example 6

### Preparation of (1S,2R,3R,4R)-4-[(naphthalen-2-yl)carbonyloxy]-3-[(1E)-3-oxo-4-phenoxybut-1-en-1-yl]-2-[(2Z)-7-oxo-7-(propan-2-yloxy)hept-2-en-1-yl]cyclopentyl naphthalene-2-carboxylate (VIb)

To a solution of compound V (0.5 g, 1.16 mmol) in CH₂Cl₂ was added 2-naphthoyl chloride (0.89 g, 4.65 mmol) and pyridine (1 mL) at 5±5° C. The mixture was stirred at 25±5° C. until compound V was consumed completely (7 h). The mixture was separated by adding MTBE (10 mL) and H₂O (10 mL). The organic layer was washed twice with 2 N HCl (10 mL), followed by NaHCO₃ (10 mL) and brine (10 mL). The organic layer was dried over MgSO₄, filtered, and evaporated to provide compound VIb. The compound crystallized with MTBE/n-Heptane as off-white solid (0.36 g, 40%). Melting point: 50-51° C. ¹H NMR (400 MHz, Chloroform-d) δ 8.65 (s, 1H), 8.40 (s, 1H), 8.12 (d, 2H), 7.96-7.85 (m, 4H), 7.77 (d, 1H), 7.70-7.58 (m, 2H), 7.55-7.45 (m, 3H), 7.36-7.18 (m, 3H), 6.95 (t, 1H), 6.88 (d, 2H), 6.45-6.30 (m, 1H), 6.0 (m, 1H), 5.41-5.49 (m, 1H), 5.25-5.40 (m, 3H), 4.99 (m, 1H), 4.72 (s, 2H), 2.95-3.05 (m, 1H), 2.65-2.82 (m, 1H), 2.41-1.82 (m, 11H), 1.91-1.56 (m, 5H), 1.23 (s, 3H), 1.21 (s, 3H). MS: m/z 756.3 [M+NH₄]⁺.

### Example 7

### Preparation of (1S,2R,3R,4R)-4-(benzoyloxy)-3-[(1E)-3-oxo-4-phenoxybut-1-en-1-yl]-2-[(2Z)-7-oxo-7-(propan-2-yloxy)hept-2-en-1-yl]cyclopentyl benzoate (VIc)

Benzoyl chloride (0.63 ml, 5.4 mmol) was added drop-wise to a solution of compound V (800 mg, 1.8 mmol) and pyridine (0.87 ml, 10.8 mmol) in DCM (8 ml) at 0±5° C., and the mixture was stirred at 25±5° C. for 4 h. The mixture was separated by adding MTBE (40 mL) and 1% NaOH (20 mL). The organic layer was washed with two portions of 2 N HCl (10 mL) twice, followed by H₂O (10 mL) and brine (10 mL). The organic layer was then dried over Na₂SO₄, filtered, evaporated, and purified by chromatography (eluent: EtOAc/Heptane=1/10) to afford compound VIc (450 mg, 40%). ¹H NMR (400 MHz, Chloroform-d) δ 8.10 (d, J=7.6 Hz, 2H), 7.92 (d, J=7.6 Hz, 2H), 7.61 (t, J=7.4 Hz, 1H), 7.53 (t, J=7.5 Hz, 1H), 7.48 (t, J=7.7 Hz, 2H), 7.34 (t, J=7.8 Hz, 2H), 7.29 (t, J=7.8 Hz, 2H), 7.12 (dd, J=15.8, 8.9 Hz, 1H), 6.98 (t, J=7.4 Hz, 1H), 6.91 (d, J=7.9 Hz, 2H), 6.63 (d, J=15.8 Hz, 1H), 5.50 (t, J=4.5 Hz, 1H), 5.41-5.33 (m, 3H), 4.96 (hept, J=6.2 Hz, 1H), 4.76 (d, J=1.2 Hz, 2H), 3.11-3.01 (m, 1H), 2.76-2.65 (m, 1H), 2.43-2.31 (m, 1H), 2.24-2.03 (m, 5H), 2.00-1.83 (m, 2H), 1.66-1.43 (m, 2H), 1.21 (s, 3H), 1.19 (s, 3H). MS: m/z 656.4 [M+NH₄]⁺.

### Example 8

### Preparation of (1S,2R,3R,4R)-4-[(4-bromophenyl)carbonyloxy]-3-[(1E)-3-oxo-4-phenoxybut-1-en-1-yl]-2-[(2Z)-7-oxo-7-(propan-2-yloxy)hept-2-en-1-yl]cyclopentyl 4-bromobenzoate (VId)

To a solution of compound V (0.5 g, 1.16 mmol) in CH₂Cl₂ was added 4-bromobenzoyl chloride (1.0 g, 4.65 mmol) and pyridine (1 mL) at 5±5° C. The mixture was stirred at 25±5° C. until compound V was consumed completely (4 h). The mixture was separated by adding MTBE (10 mL) and H₂O (10 mL). The organic layer was washed twice with 2 N HCl (10 mL), followed by NaHCO₃ (10 mL) and brine (10 mL). The organic layer was dried over MgSO₄, filtered, evaporated, and purified by chromatography (eluent: EtOAc/Heptane=1/5) to afford compound VId (0.5 g, 56%) as pale-yellow oil. ¹H NMR (400 MHz, Chloroform-d) δ 7.90 (d, J=8.6 Hz, 2H), 7.72 (d, J=8.6 Hz, 2H), 7.60 (d, J=8.6 Hz, 2H), 7.47 (d, J=8.6 Hz, 2H), 7.31-7.22 (m, 2H), 7.06 (dd, J=15.8, 8.9 Hz, 1H), 6.96 (t, J=7.8 Hz, 1H), 6.87 (d, J=7.8 Hz, 2H), 6.60 (d, J=15.8 Hz, 1H), 5.41-5.49 (m, 1H), 5.25-5.40 (m, 3H), 4.94 (dp, J=12.6, 6.3 Hz, 1H), 4.72 (d, J=1.6 Hz, 2H), 3.06-2.92 (m, 1H), 2.77-2.62 (m, 1H), 2.41-1.82 (m, 9H), 1.91-1.56 (m, 3H), 1.20 (s, 3H), 1.18 (s, 3H). MS: m/z 814.1 [M+NH₄]⁺.

### Example 9

### Preparation of (1S,2R,3R,4R)-4-[(4-methoxyphenyl)carbonyloxy]-3-[(1E)-3-oxo-4-phenoxybut-1-en-1-yl]-2-[(2Z)-7-oxo-7-(propan-2-yloxy)hept-2-en-1-yl]cyclopentyl 4-methoxybenzoate (VIe)

To a solution of compound V (0.5 g, 1.16 mmol) in CH₂Cl₂ was added 4-methoxybenzoyl chloride (0.8 g, 4.65 mmol) and pyridine (1 mL) at 5±5° C. The mixture was stirred at 25±5° C. until compound V was consumed completely (24 h). The mixture was separated by adding MTBE (10 mL) and H₂O (10 mL). The organic layer was washed twice with 2 N HCl (10 mL), followed by NaHCO₃ (10 mL) and brine (10 mL). The organic layer was dried over MgSO₄, filtered, evaporated, and purified by chromatography (eluent: EtOAc/Heptane=1/3) to afford compound VIe (0.2 g, 25%) as pale-yellow oil. ¹H NMR (400 MHz, Chloroform-d) δ 8.04 (m, 2H), 7.87 (m, 2H), 7.28 (m, 2H), 7.20-6.75 (m, 8H), 6.62 (m, 1H), 5.49-5.25 (m, 4H), 4.99 (m, 1H), 4.76 (m, 2H), 2.95-3.05 (m, 1H), 2.65-2.82 (m, 1H), 2.41-1.82 (m, 9H), 1.91-1.56 (m, 3H), 1.20 (s, 3H), 1.18 (s, 3H). MS: m/z 716.4 [M+NH₄]⁺.

### Example 10

### Preparation of propan-2-yl (5Z)-7-[(1R,2R,3R,5S)-3,5-bis(acetyloxy)-2-[(1E)-3-oxo-4-phenoxybut-1-en-1-yl]cyclopentyl]hept-5-enoate (VIf)

To a solution of compound V (10 g) in DCM (50 mL) was added triethylamine and DMAP, followed by a solution of Ac₂O (6.0 g, 2.5 eq.) in DCM (20 mL) at 25° C. The mixture was stirred at 25° C. for 1 hr prior to addition of saturated NH₄Cl (50 mL). The separated aqueous layer was extracted with DCM (50 mL), and the organic layers were combined and washed with saturated NaHCO₃ (40 mL) and brine (40 mL). The organic layer was dried over MgSO₄, filtered, evaporated, and purified by chromatography (eluent: EtOAc/Heptane=1/3) to give compound VIf (8.5 g, 70%). ¹H NMR (300 MHz, Chloroform-d) δ 7.38-7.21 (m, 2H), 7.08-6.83 (m, 4H), 6.50 (d, J=15.8 Hz, 1H), 5.44-5.18 (m, 2H), 5.15-5.08 (m, 1H), 5.04-4.94 (m, 2H), 4.72 (s, 2H), 2.84-2.69 (m, 1H), 2.69-2.47 (m, 1H), 2.31-1.92 (m, 11H), 1.91-1.56 (m, 5H), 1.23 (s, 3H), 1.21 (s, 3H). MS: m/z 532.3 [M+NH₄]⁺.

### Example 11

### Preparation of (1S,2R,3R,4R)-3-[(1E)-3,3-difluoro-4-phenoxybut-1-en-1-yl]-2-[(2Z)-7-oxo-7-(propan-2-yloxy)hept-2-en-1-yl]-4-[(4-phenylphenyl)carbonyloxy]cyclopentyl 4-phenylbenzoate (VIIa)

DeoxoFluor (2.3 g, 9.32 mmol based on 90% purity) was added drop wise to a solution of compound VIa (1.9 g, 2.33 mmol) in THF (1.9 mL) at 5±5° C. Care was taken because DeoxoFluor can generate corrosive HF. The mixture was warmed to 37±3° C. and stirred until the compound VIa was consumed completely (1 day). The reaction mixture was cooled to 25±5° C. and poured into chilled saturated NaHCO₃ (20 mL, 5±5° C.). EtOAc (20 mL) was added and the mixture was stirred for 5 min. The separated organic layer was washed with saturated NaHCO₃ and brine, dried over MgSO₄, filtered, and evaporated to provide compound VIIa. The crude compound crystallized with THF/n-heptane as off-white solid (600 mg, 31%). Melting point: 70-71° C. ¹H NMR (400 MHz, Chloroform-d) δ 8.18 (d, J=8.0 Hz, 2H), 7.98 (d, J=8.1 Hz, 2H), 7.70 (d, J=8.1 Hz, 2H), 7.64 (d, J=7.8 Hz, 2H), 7.56-7.37 (m, 10H), 7.28 (t, J=8.0 Hz, 3H), 7.00 (t, J=7.4 Hz, 1H), 6.92 (d, J=8.5 Hz, 2H), 6.41-6.30 (m, 1H), 6.08-5.94 (m, 1H), 5.53 (t, J=4.4 Hz, 1H), 5.49-5.39 (m, 2H), 5.39-5.32 (m, 1H), 4.97 (hept, J=6.2 Hz, 1H), 4.25 (t, J=11.8 Hz, 1H), 3.11-3.00 (m, 1H), 2.78-2.66 (m, 1H), 2.56-2.22 (m, 3H), 2.21-1.90 (m, 6H), 1.62-1.52 (m, 2H), 1.20 (s, 3H), 1.19 (s, 3H). ¹⁹F NMR (376 MHz, decoupled, CDCl₃) δ −102.30 (d, J_{FF}=256.4 Hz), −104.21 (d, J_{FF}=256.5 Hz). MS: m/z 830.3 [M+NH₄]⁺.

### Example 12

### Preparation of (1S,2R,3R,4R)-3-[(1E)-3,3-difluoro-4-phenoxybut-1-en-1-yl]-4-[naphthalen-2-yl)carbonyloxy]-2-[(2Z)-7-oxo-7-(propan-2-yloxy)hept-2-en-1-yl]cyclopentyl naphthalene-2-carboxylate (VIIb)

DeoxoFluor (0.2 g) was added drop wise to a solution of compound VIb (200 mg, 0.27 mmol) in toluene (1 mL). The mixture was stirred at 25° C. for 1 day. The reaction mixture was quenched with sat. NaHCO₃ followed by addition of EtOAc. The separated organic layer was washed with saturated NaHCO₃ and brine, and dried over MgSO₄, filtered, and evaporated to provide compound VIIb. The compound crystallized with MTBE/n-Heptane as off-white solid (80 mg, 40%). Melting point: 53-54° C. ¹H NMR (400 MHz, Chloroform-d) δ 8.65 (s, 1H), 8.40 (s, 1H), 8.12 (d, J=9.6 Hz 2H), 7.96-7.85 (m, 4H), 7.77 (d, J=2.4 Hz, 1H), 7.70-7.58 (m, 2H), 7.55-7.45 (m, 3H), 7.36-7.18 (m, 3H), 6.95 (t, J=6.2 Hz, 1H), 6.88 (d, J=5.6 Hz, 2H), 6.45-6.30 (m, 1H), 6.0 (m, 1H), 5.41-5.49 (m, 1H), 5.25-5.40 (m, 3H), 4.99 (m, 1H), 4.72 (s, 2H), 2.95-3.05 (m, 1H), 2.65-2.82 (m, 1H), 2.41-1.82 (m, 9H), 1.91-1.56 (m, 3H), 1.23 (s, 3H), 1.21 (s, 3H). ¹⁹F NMR (374 MHz, Chloroform-d) δ −102.35 (dq, J_{FF}=256 Hz, J_{FH}=11.2 Hz), −104.36 (dq, J_{FF}=256.0 Hz, J_{FH}=11.2 Hz). MS: m/z 778.3 [M+NH₄]⁺.

### Example 13

### Preparation of (1S,2R,3R,4R)-4-(benzoyloxy)-3-[(1E)-3,3-difluoro-4-phenoxybut-1-en-1-yl]-2-[(2Z)-7-oxo-7-(propan-2-yloxy)hept-2-en-1-yl]cyclopentyl benzoate (VIIc)

A solution of compound VIc (400 mg, 0.6 mmol) and EtOH (17 μL) in THF (2 mL) was added drop-wise to DeoxoFluor (0.49 ml, 2.4 mmol based on 90% purity) at 0±5° C. The mixture was warmed to 45±3° C. and stirred for 36 h. The reaction mixture was cooled to 25±5° C. and poured into chilled saturated NaHCO₃ (20 mL, 5±5° C.). MTBE (20 mL) was added and the mixture was stirred for 5 min. The separated organic layer was washed with saturated NaHCO₃ and brine, dried over MgSO₄, filtered, and evaporated to provide compound VIIc. The crude product was purified by chromatography (eluent: EtOAc/Heptane=1/5) as colorless oil (180 mg, 43%). ¹H NMR (400 MHz, Chloroform-d) δ 8.10 (d, J=8.1 Hz, 2H), 7.93 (d, J=8.0 Hz, 2H), 7.61 (t, J=6.9 Hz, 1H), 7.59-7.35 (m, 3H), 7.35-7.18 (m, 4H), 7.00 (t, J=7.4 Hz, 1H), 6.90 (d, J=8.5 Hz, 1H), 6.39-6.23 (m, 1H), 6.04-5.85 (m, 1H), 5.63-5.21 (m, 4H), 4.96 (hept, J=6.2 Hz, 1H), 4.22 (t, J=11.7 Hz, 2H), 3.16-2.93 (m, 1H), 2.93-2.62 (m, 1H), 2.45-2.26 (m, 2H), 2.18-1.81 (m, 6H), 1.60-1.46 (m, 2H), 1.22 (s, 3H), 1.20 (s, 3H). ¹⁹F NMR (376 MHz, decoupled, CDCl₃) δ −102.41 (d, J_{FF}=256.4 Hz), −104.12 (d, J_{FF}=256.4 Hz). MS: m/z 678.3 [M+NH₄]⁺.

### Example 14

### Preparation of (1S,2R,3R,4R)-4-[(4-bromophenyl)carbonyloxy]-3-[(1E)-3,3-difluoro-4-phenoxybut-1-en-1-yl]-2-[(2Z)-7-oxo-7-(propan-2-yloxy)hept-2-en-1-yl]cyclopentyl 4-bromobenzoate (VIId)

To a solution of compound VId (230 mg, 0.3 mmol) in toluene (1 mL) was added Fluolead (0.23 g) and hydrogen fluoride-pyridine (65-70%, 0.028 mL). The mixture was stirred at 25° C. for 5 h. The reaction mixture was quenched with MeOH/MTBE followed by addition of sat. NaHCO₃. The separated organic layer was washed with saturated NaHCO₃ and brine, and dried over MgSO₄, filtered, evaporated, and purified by chromatography (eluent: EtOAc/heptane=1/8) to afford compound VIId (110 mg, 44%) as pale-yellow oil. ¹H NMR (400 MHz, Chloroform-d) δ 7.91 (d, J=8.4 Hz, 2H), 7.72 (d, J=8.5 Hz, 2H), 7.59 (d, J=8.4 Hz, 2H), 7.43 (d, J=8.4 Hz, 2H), 7.26 (t, J=7.3 Hz, 2H), 6.98 (t, J=7.3 Hz, 1H), 6.85 (d, J=8.5 Hz, 2H), 6.33-6.19 (m, 1H), 6.02-5.86 (m, 1H), 5.41-5.46 (m, 1H), 5.41-5.30 (m, 2H) 5.21-5.27 (m, 1H), 4.94 (dp, J=12.6, 6.3 Hz, 1H), 4.18 (dd, J=18.0, 7.4 Hz, 2H), 2.96-2.90 (m, 1H), 2.77-2.63 (m, 1H), 2.41-1.79 (m, 9H), 1.91-1.56 (m, 3H), 1.23 (s, 3H), 1.20 (s, 3H). ¹⁹F NMR (374 MHz, Chloroform-d) δ—102.35 (dq, J_{FF}=256 Hz, J_{FH}=11.2 Hz), −104.36 (dq, J_{FF}=256.0 Hz, J_{FH}=11.2 Hz). MS: m/z 836.1 [M+NH₄]⁺.

### Example 15

### Preparation of (1S,2R,3R,4R)-3-[(1E)-3,3-difluoro-4-phenoxybut-1-en-1-yl]-4-[(4-methoxyphenyl)carbonyloxy]-2-[(2Z)-7-oxo-7-(propan-2-yloxy)hept-2-en-1-yl]cyclopentyl 4-methoxybenzoate (VIIe)

To a solution of compound VIe (100 mg, 0.14 mmol) in toluene (1 mL) was added Fluolead (0.1 g) and hydrogen fluoride-pyridine (65-70%, 0.013 mL). The mixture was stirred at 25° C. for 6 h. The reaction mixture was quenched with MeOH/MTBE followed by addition of sat. NaHCO₃. The separated organic layer was washed with saturated NaHCO₃ and brine, and dried over MgSO₄, filtered, evaporated, and purified by chromatography (eluent: EtOAc/heptane=1/5) to afford compound VIIe (20 mg, 20%) as pale-yellow oil. ¹H NMR (400 MHz, Chloroform-d) δ 8.02 (d, J=8.8 Hz, 2H), 7.85 (d, J=8.8 Hz, 2H), 7.30-7.21 (m, 3H), 6.98 (d, J=7.4 Hz, 2H), 6.94 (t, J=7.8 Hz, 1H), 6.87 (d, J=7.9 Hz, 2H), 6.78 (d, J=8.9 Hz, 2H), 6.31-6.24 (m, 1H), 6.00-5.86 (m, 1H), 5.48-5.42 (m, 3H), 5.41-5.38 (m, 1H), 4.95 (dp, J=12.6, 6.3 Hz 1H), 4.19 (t, J=12.9 Hz, 2H), 3.87 (s, 3H), 3.81 (s, 3H), 3.01-2.95 (m, 1H), 2.62-2.56 (m, 1H), 2.42-1.80 (m, 9H), 1.91-1.56 (m, 3H), 1.23 (s, 3H), 1.21 (s, 3H). ¹⁹F NMR (374 MHz, Chloroform-d) δ −102.36 (dq, J_{FF}=254.6 Hz, J_{FH}=11.2 Hz), −104.34 (dq, J_{FF}=254.6 Hz, J_{FH}=11.2 Hz). MS: m/z 554.4 [M+NH₄]⁺. MS: m/z 738.3 [M+NH₄]⁺.

### Example 16

### Preparation of propan-2-yl (5Z)-7-[(1R,2R,3R,5S)-3,5-bis(acetyloxy)-2-[(1E)-3,3-difluoro-4-phenoxybut-1-en-1-yl]cyclopentyl]hept-5-enoate

A solution of VIf (0.78 g) in DCM (1 mL) was added drop-wise to a plastic bottle charged with Deoxo-Fluor (4 eq, 50% in THF) at 0-5° C. The reaction mixture was stirred at 35-40° C. for 26 h, and then cooled to 25° C. and poured into a chilled (5° C.) mixture of saturated NaHCO₃ (10 mL) and EtOAc (10 mL). The separated organic layer was washed with saturated NaHCO₃ (10 mL) and brine, dried over MgSO₄, filtered, and evaporated to afford VIIf (480 mg, 61%). The crude product was purified by chromatography (eluent: EtOAc/Heptane=1/4). ¹H NMR (300 MHz, Chloroform-d) δ 7.34-7.27 (m, 2H), 7.04-6.96 (m, 1H), 6.94-6.88 (m, 2H), 6.19-6.02 (m, 1H), 5.93-5.74 (m, 1H), 5.44-5.24 (m, 2H), 5.11 (t, J=5.1 Hz, 1H), 5.05-4.89 (m, 2H), 4.18 (t, 2H), 2.76-2.48 (m, 2H), 2.31-1.89 (m, 1H), 1.83-1.59 (m, 5H), 1.23 (s, 3H), 1.21 (s, 3H). ¹⁹F NMR (282 MHz, Chloroform-d) δ −102.95 (dq, J_{FF}=258.0 Hz, J_{FH}=11.4 Hz), −104.38 (dq, J_{FF}=258.0 Hz, J_{FH}=11.4 Hz). MS: m/z 554.4 [M+NH₄].

### Example 17

### Preparation of Tafluprost (I)

To a solution of compound VIIa (47 mg, 0.058 mmol) in THF/IPA (v/v=1/1, 1.0 mL) was added iPrONa (10 mg, 0.11 mmol) at 25±5° C., and the resulting mixture was stirred for 1 h. AcOH (0.2 mmol) and saturated NaHCO₃ were added, and the mixture was filtered to remove precipitated solid (PhBzOiPr and PhBzOH). The filter cake was washed with IPA and the filtrate was extracted with MTBE (1.0 mL). The organic layer was washed with H₂O (1.0 mL) and brine, dried over MgSO₄, filtered, and evaporated to provide tafluprost (I) (20 mg, 76%). The product was purified by chromatography (eluent: EtOAc/Heptane=1/1). ¹H NMR (300 MHz, Chloroform-d) δ 7.39-7.22 (m, 2H), 7.05-6.95 (m, 1H), 6.95-6.86 (m, 2H), 6.10 (ddt, J=15.7, 9.0, 2.4 Hz, 1H), 5.89-5.70 (m, 1H), 5.48-5.29 (m, 2H), 4.99 (hept, J=6.2 Hz, 1H), 4.19 (t, J=11.6 Hz, 3H), 4.02 (s, 1H), 2.81 (s, 1H), 2.62 (s, 1H), 2.47 (td, J=9.8, 4.3 Hz, 1H), 2.40-2.20 (m, 3H), 2.20-1.96 (m, 4H), 1.88-1.78 (m, 1H), 1.71-1.52 (m, 3H), 1.23 (s, 3H), 1.21 (s, 3H). ¹⁹F NMR (282 MHz, Chloroform-d) δ −100.76 (dq, J_{FF}=250.0 Hz, J_{FH}=12.1 Hz), −102.21 (dq, J_{FF}=250.0 Hz, J_{FH}=12.1 Hz). MS: m/z 453.3 [M+H]⁺.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A process for preparing a prostaglandin analog of formula I 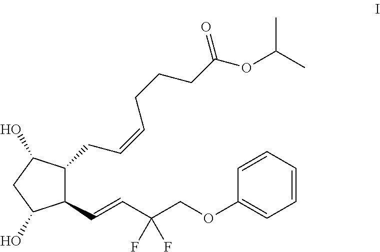 [SMILES: CC(C)OC(=O)CCC/C=C\C[C@@H]1[C@@H](/C=C/C(F)(F)COc2ccccc2)[C@H](O)C[C@@H]1O]
comprising:
a) contacting a compound of formula II 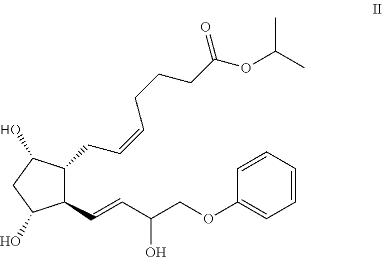 [SMILES: CC(C)OC(=O)CCC/C=C\C[C@@H]1[C@@H](/C=C/C(O)COc2ccccc2)[C@H](O)C[C@@H]1O]
with a compound selected from the group consisting of a boronic acid, a boronate ester, and an aminoborane, under conditions sufficient to provide a compound of formula III 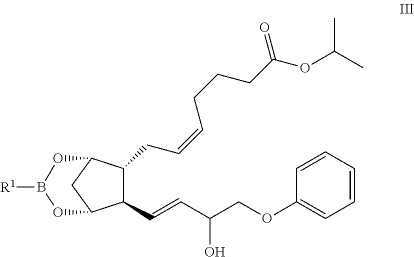
wherein R¹ is selected from the group consisting of an optionally substituted alkyl group, an optionally substituted aryl group, or a polystyrene support;
b) converting the compound of formula III to a compound of formula V 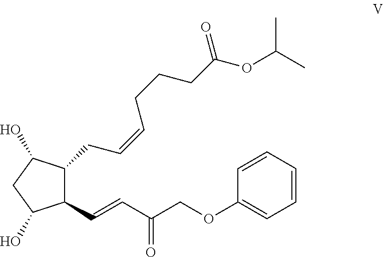 [SMILES: CC(C)OC(=O)CCC/C=C\C[C@@H]1[C@@H](/C=C/C(=O)COc2ccccc2)[C@H](O)C[C@@H]1O]
using an oxidant and a subsequent basic wash solution;
c) converting the compound of formula V to a compound of formula VI 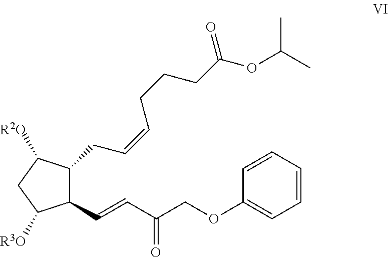
wherein R² and R³ are independently selected from hydroxy protecting groups or are taken together to form a single hydroxy protecting group;
d) fluorinating the compound of formula VI to give a compound of VII 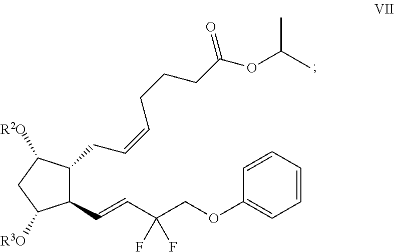
and
e) removing R² and R³ from the compound of VII under basic conditions to provide the compound of formula I.

2. The process of claim 1, wherein the oxidant is selected from the group consisting of PCC, PCC/Al₂O₃, Jones reagent, Collins reagent, PDC, DMSO/DCC, DMSO/SO₃-pyridine, DMSO/(COCl)₂, DMSO/TFAA, DMSO/Ac₂O, Dess-Martin periodinane, IBX, and TEMPO.

3. The process of claim 1, wherein the basic wash solution is selected from the group consisting of NaOH (aq), Na₂CO₃ (aq), NaHCO₃ (aq), K₂CO₃ (aq), LiOH (aq), and KOH (aq).

4. The process of claim 1, wherein the boronic acid is selected from the group consisting of an alkyl boronic acid, a phenyl boronic acid, a polymer-supported boronic acid, and a diboronic acid.

5. The process of claim 1, wherein R¹ is selected from the group consisting of alkyl, phenyl, and a polystyrene support.

6. The process of claim 5, wherein R¹ is phenyl.

7. The process of claim 1, wherein the boronate ester is selected from the group consisting of an alkyl dialkoxyl borane, substituted or unsubstituted phenyl boronate ester, trialkyl boronate or 1,1,2,2-tetraalkoxy-diborane and polystyrene supported boronate dialkyl ester.

8. The process of claim 1, wherein the aminoborane is selected from the group consisting of an alkyldiaminoborane, tris(dialkylamino)borane and tetrakis(dialkylamino)diboron.

9. The process of claim 1, wherein R² and R³ are independently selected from the group consisting of acetate, chloroacetate, dichloroacetate, trichloroacetate, trifluoroacetate, pivaloate, benzoate, p-methoxybenzoate, p-bromobenzoate, p-chlorobenzoate, Fmoc, Cbz, 2-naphthalenecarboxylate and 4-phenyl benzoate.

10. The process of claim 9, wherein R² and R³ are 4-phenyl benzoate.

11. The process of claim 1, wherein step (d) comprises a fluorination reagent selected from the group consisting of diethylaminosulfur trifluoride, bis(2-methoxyethyl)aminosulfur trifluoride, diethylaminodifluorosulfinium tetrafluoroborate, morpholinoifluorosulfinium tetrafluoroborate, and 4-tert-butyl-2,6-dimethylphenylsulfur trifluoride.

12. The process of claim 1, wherein step (e) comprises a base selected from the group consisting of Na₂CO₃, NaHCO₃, K₂CO₃, NaOH, LiOH, KOH, NaOMe, NaOEt, NaOⁱPr, LiO^{t}Bu, NaO^{t}Bu, KO^{t}Bu, Et₃N, and DBU.

13. A compound of formula Ma

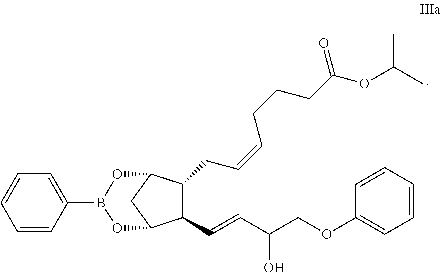 [SMILES: CC(C)OC(=O)CCC/C=C\C[C@@H]1[C@@H](/C=C/C(O)COc2ccccc2)[C@H]2C[C@@H]1OB(c1ccccc1)O2]

14. A compound of formula VI 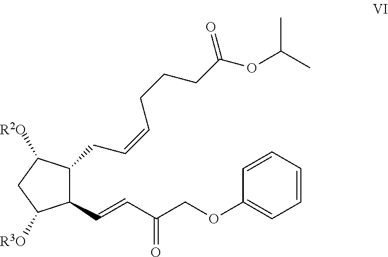
wherein R² and R³ are independently selected from hydroxy protecting groups or are taken together to form a single hydroxy protecting group.

15. The compound of claim 14, wherein R² and R³ are independently selected from the group consisting of acetate, chloroacetate, dichloroacetate, trichloroacetate, trifluoroacetate, pivaloate, benzoate, p-methoxybenzoate, p-bromobenzoate, p-chlorobenzoate, Fmoc, Cbz, 2-naphthalenecarboxylate and 4-phenyl benzoate.

16. The compound of claim 15, wherein R² and R³ are 4-phenyl benzoate.

17. A compound of formula VII 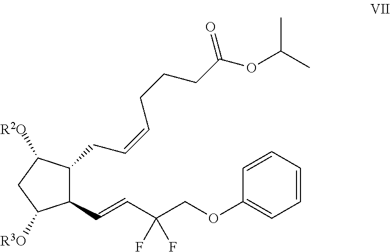
wherein R² and R³ are independently selected from hydroxy protecting groups or are taken together to form a single hydroxy protecting group.

18. The compound of claim 17, wherein R² and R³ are independently selected from the group consisting of acetate, chloroacetate, dichloroacetate, trichloroacetate, trifluoroacetate, pivaloate, benzoate, p-methoxybenzoate, p-bromobenzoate, p-chlorobenzoate, Fmoc, Cbz, 2-naphthalenecarboxylate and 4-phenyl benzoate.

19. The compound of claim 18, wherein R² and R³ are 4-phenyl benzoate

20. The compound of claim 19, having less than 1% impurity.
